SEQUENCE LISTING (7 sequences)

SEQ ID NO: 1 - DNA, 579 bp, Rhopalosiphium padi virus
gataaaagaacctataatcccttcgcacaccgcgtcacaccgcgctatatgctgctcattaggaattacggctccttttttgtggatacaatctcttgtatacgatatacttattgttaatttcattgacctttacgcaatcctgcgtaaatgctggtatagggtgtacttcggatttccgagcctatattggttttgaaaggacctttaagtccctactatactacattgtactagcgtaggccacgtaggcccgtaagatattataactattttattatattttattcaccccccacattaatcccagttaaagctttataactataagtaagccgtgccgaaacgttaatcggtcgctagttgcgtaacaactgttagtttaattttccaaaatttatttttcacaatttttagttaagattttagcttgccttaagcagtctttatatcttctgtatattattttaaagtttataggagcaaagttcgctttactcgcaatagctattttatttattttaggaatattatcacctcgtaattatttaattataacattagctttatctatttata

SEQ ID NO: 2 - DNA, 28 bp, Artificial Sequence
  Chemically synthesized PCR primer
gccgatatctagttaattaatagtaatc

SEQ ID NO: 3 - DNA, 29 bp, Artificial Sequence
  Chemically synthesized PCR primer
agcgctagcggatctgacggttcactaaa

SEQ ID NO: 4 - DNA, 42 bp, Artificial Sequence
  Chemically synthesized PCR primer
atataagatctccaccatgctgctgctgctgctgctgctggg

SEQ ID NO: 5 - DNA, 34 bp, Artificial Sequence
  Chemically synthesized PCR primer
aattcagatctggtgtctgctcgaagcggccggc

SEQ ID NO: 6 - DNA, 20 bp, Artificial Sequence
  Chemically synthesized PCR primer
acgacttcttcaagtccgcc

SEQ ID NO: 7 - DNA, 20 bp, Artificial Sequence
  Chemically synthesized PCR primer
tgctcaggtagtggttgtcg

(19) United States
(12) Patent Application Publication
(10) Pub. No.: US 2011/0207210 A1
(43) Pub. Date: Aug. 25, 2011

(54) Isolated or synthesized Rhopalosiphum padi virus polynucleotides having a promotor activity

(75) Inventors: Tzong-Yuan Wu, Taoyuan County (TW); Ying-Ju Chen, Taoyuan County (TW); Chao-Yi Teng, Taoyuan County (TW); Yi-Ting Lin, Taoyuan County (TW); Yu-Jie Chen, Taoyuan County (TW); Yi-Jane Wu, Taoyuan County (TW); Seng-Chi Chen, Taoyuan County (TW)

(73) Assignee: Chung Yuan Christian University, Jhongli City (TW)

(21) Appl. No.: 12/322,181

(22) Filed: Jan. 31, 2009

(51) Int. Cl. C12N 15/866

(52) U.S. Cl. 435/320.1

(57) ABSTRACT

Provided herein are isolated or synthesized Rhopalosiphum padi virus (RhPV) nucleic acids having promotor activities, and vectors containing the isolated or synthesized RhPV nucleic acids for gene delivery in insect expression systems.

### BACKGROUND

1. Field of the Invention

This invention in general relates to an isolated or synthesized Rhopalosiphum padi virus (RhPV) nucleic acid having a promotor activity, and to vectors containing the isolated or synthesized RhPV nucleic acid.

2. Description of related arts

Rhopalosiphum padi virus (RhPV) is an insect virus that infects a narrow range of aphid species of the Rhopalosiphum and Schizaphis families. Upon infecting a host cell, RhPV translation proceeds in a cap-independent manner by use of an internal ribosomal entry site (IRES) within its 5′ untranslated region (UTR). The 5′ UTR of RhPV is reported to be 579 nucleotide-long, and possesses cross-kingdom IRES activity that functions in mammalian-, plant-, or insect-derived in vitro translation system (Terenin I M et al., Mol Cell Biol 2005 25:7879-7888). Therefore, IRES element has been successfully introduced between 2 cistrons to construct bicistronic vectors for simultanelusly co-expressing two different proteins, one by cap-dependent mechanism and the other by cap-independent mechanism (i.e., IRES-dependent translation).

Inventors of this study unexpectedly identify that RhPV IRES element may act, not only as an IRES element, but also as a promotor, for initiating gene expression operably linked thereto in an baculovirus infected insect expression system. This promotor activity of RhPV is believed to be conferred by the six TAAG motifs within the RhPV IRES sequence, and therefore RhPV IRES is useful for constructing vectors for gene delivery in insect systems.

### SUMMARY

As embodied and broadly described herein, the invention features an isolated or synthesized Rhopalosiphum padi virus (RhPV) polynucleotide that is capable of initiating transcription of an operably linked nucleic acid sequence in an insect expression system; and a vector comprising the same.

The present inventors have found that the 579 nucleotide (nt)-long 5′-UTR of Rhopalosiphum padi virus, particularly the sequence set forth in SEQ ID NO: 1 of the sequence listing has a specific promotor activity in an insect expression system, and that it is useful as a promotor for constructing a vector for gene delivery.

Therefore, the present invention relates to a nucleic acid isolated from 5′-UTR of Rhopalosiphum padi virus having a promotor activity and a polynucleotide sequence set forth in SEQ ID NO: 1 of the sequence listing, or a fragment thereof. The isolated nucleic acid comprises 6 TAAG motifs and is capable of initiating transcription of an operably linked nucleic acid sequence in insect cells, such as S. frugiperda IPBL-Sf21 insect cells. The isolated nucleic acid also act as an internal ribosomal entry site (IRES) and initiates a cap-independent translation of a nucleic acid operably linked thereto in mammalian cells.

Furthermore, the present invention relates to a vector comprising a promotor sequence isolated from RhPV, the promotor sequence is capable of initiating transcription of an operably linked nucleic acid in insect cells, and has a a polunecleotide sequence at least 90% identical to the sequence set forth in SEQ ID NO.: 1 of the sequence listing. In one preferred example, the vector is baculovirus, and the insect cells are S. frugiperda IPBL-Sf21 insect cells.

The details of one or more embodiments of the invention are set forth in the accompanying description and drawings below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The invention will be illustrated with respect to the accompanying figures and examples, which serve to illustrate this invention but are not binding thereon, wherein:

FIG. 1 is a schematic presentation of bicistronic constructs of plasmids and recombinant baculoviruses produced in accordance with procedures described in this invention. (A) The positive control plasmid pD-E in which the DsRed and EGFP genes were fused in-frame. (B) The negative control plasmid pD-E/O in which the DsRed and EGFP genes were fused out-frames. (C) pCMV-DRhirE, the RhPV 5′UTR-IRES is located between the DsRed and EGFP genes. (D) Construction of the plasmid, pCMV-D-Rhir-SE, used to quantify RhPV IRES activity. And schematic presentation of the recombinant baculovirus, (E) vAcCMV-DRhirE; (F) vAc-DRhirE and (G) vAcΔ-DRhirE. (H) Construction of the plasmid, pIE2-DRhirE, used to analysis RhPV IRES activity in insect cells without the baculovirus infection. CMV, human cytomegalovirus immediately early promoter; DsRed, red fluorescent protein gene; IRES, element of RhPV IRES; EGFP, enhanced green fluorescent protein gene; SEAP-EGFP, SEAP in-frame-fused with the EGFP gene. ETL, AcMNPV early to late promoter; LacZ, β-galactosidase reporter gene; pH, polyhedrin promoter; IE2, the early promoter derived from Orgyia pseudotsugata multiple nucleopolyhedrovirus (OpMNPV). The stop indicates the translational termination signal.

FIG. 2 are photographs illustrating the RhPV IRES activity in mammalian cells. CHO-k1 cells (9×10⁴˜10⁵ cells per well seeded in a 24-well plate) were transfected with pD-E (A) or pCMV-DRhirE (B) and observed at 2 days after transfection under fluorescence microscopy. Pictures were taken in the same is field with a conventional rhodamine channel (Red) with a 510/560-nm filter set and an FITC channel (Green) with a 450/490-nm filter set. Both pictures were taken at the same exposure time of 360 ms. Scale bar, 20 μm.

FIG. 3 is a bar graph illustrating a comparison of the efficiency of RhPV IRES in driving translation in five different cell lines. CHO, HeLa, HepG2, COS-1, and MDCK cells transfected with 1 μg of the pCMV-DRhirSE plasmid as described in “Materials and Methods”. At 48 h after transfection, SEAP activities in the medium were determined. All data are normalized for transfection efficiency by calibration through red fluorescent cells and presented as the mean±SD of three independent experiments.

FIG. 4 are photographs illustrating fluorescence patterns emitted from recombinant virus vAcCMV-DRhirE-infected Sf21 cells and transduced COS-1 cells and U2OS cells. (A) vAcCMV-DRhirE-infected Sf21 cells (MOI=5) were stained with X-Gal at 48 h after infection (left panel) and observed under fluorescence microscopy at 5 days post-infection (dpi) (right panel). The green fluorescence picture was taken with the FITC channel (green) with a 450/490-nm filter set. Scale bar, 80 μm. (B) COS-1 and (C) U2OS cells (5×10³ cells seeded in a 24-well plate) were transduced with vAcCMV-DRhirE at a multiplicity of infection (MOI) of 150 in the presence of 10 mM sodium butyrate and observed under fluorescence microcopy. Pictures were taken in the same field with a conventional rhodamine channel (Red) with a 510/560-nm filter set and an FITC channel (Green) with a 450/490-nm filter set. Both pictures were taken at the same exposure time of 360 ms. Scale bar, 20 μm.

FIG. 5 illustrates RhPV IRES containing six TAAG motifs and mediating transcription in baculovirus infected Sf21 cells. (A) Northern blot analysis of RNA transcripts derived from vAcCMV-DRhirE infected Sf21 cells. Total RNA transcripts extracted from vAcCMV-DRhirE infected Sf21 cells at 4 days post-infection (lane 1) and uninfected Sf21 cells (lane 2). A DIG-labeled probe specific for the EGFP sequence detected only a single species of RNA of 0.8 kb in vAcCMV-DRhirE infected Sf21 cells lane 1 (arrow). The RNA markers are indicated in the right of lane 2 (kb). (B) The 579 nucleotides of the RhPV IRES with the six TAAG motifs are in yellow. (C) Northern blotting of RNA transcripts extracted from vAcD-Crir-E (lane 1) and vAcD-Rhir-E (lane 2) infected Sf21 cells at 4 d post-infection, RNA was detected with a DIG-labeled probe specific for the EGFP sequence, as described in “Material and Methods”. The predicted bicistronic transcript (about 2.2 kb) and monocistronic transcript (about 0.8 kb) are indicated (arrow).

FIG. 6 illustrates results of the promoterless assay of the RhPV IRES in recombinant baculovirus-infected Sf21 cells. Sf21 cells (2×10⁵ cells per well seeded in a 24-well plate) were infected with (A) vAc-DRhirE or (B) vAcΔ-DRhirE at a multiplicity of infection (MOI) of 2 and observed at 4 days post inoculation (dpi) under fluorescence microscopy. (C) Western blot analysis of cell extracts of these viruses (MOI=5) infected Sf21 cells probed with DsRed (left panel) and EGFP (right panel) specific antibodies. Lane 1, the vAc-DRhirE infected cells; Lane 2, vAcCMV-DRhirE infected cells; lane 3, vAcΔ-DRhirE infected cells. The detected protein bands are DsRed (leftr panel) and EGFP (right panel) as indicated by the arrow. The signal presented below the DsRed proteins may be the degraded DsRed proteins (lane 1, left panel). Pictures were taken in the same field with a conventional rhodamine channel (Red) with a 510/560-nm filter set and an FITC channel (Green) with a 450/490-nm filter set. Both pictures were taken at the same exposure time of 260 ms. Scale bar, 40 μm.

FIG. 7 illustrates the fluorescence patterns of pIE2-DRhirE transfected cells, in which red fluorescence, but not green fluorescence, was observed. Sf21 cells transfected with 1 μg of the pIE2-DRhirE plasmid as described in “Materials and Methods” and observed at 2 days after transfection under fluorescence microscopy. Pictures were taken in the same field with a conventional rhodamine channel (Red) with a 510/560-nm filter set and an FITC channel (Green) with a 450/490-nm filter set. Both pictures were taken at the same exposure time of 260 ms. Scale bar, 40 μm.

### DETAILED DESCRIPTION OF THE INVENTION

Unless otherwise indicated, all terms used herein have the same meaning as they would to one skilled in the art and the practice of this invention will employ, conventional techniques of microbiology and recombinant DNA technology, which are within the knowledge of those of skill of the art.

The term “promotor activity” refers to a nucleic acid having the ability in being recognized by RNA polymerase and other proteins to initiative transcription of a heterologus DNA operably linked thereto.

The term “operably linked” as used herein refers to linkage of the promotor 5′ relative to a nucleic acid sequence such that the promotor mediates transcription of the linked nucleic acid sequence. It is understood that the promotor sequence also includes transcribed sequence between transcriptional start and translational start codon.

The term “vector” refers to expression systems, nucleic acid-based shuttle vehicles, nucleic acid molecules adapted for nucleic acid delivery, and autonomous self-replicating circular DNA (e.g., plasmids, cosmids, phagemids, and the like). Where a recombinant microorganism or cell culture is described as hosting a “vector”, this includes extrachromosomal circular DNA, DNA that has been incorporated into the host chromosomes, or both.

The term “isolated” when referring to nucleic acid sequences, refers to subject nucleic acids that do not contain the naturally occurring adjacent counterpart sequences. For example, the IRES RhPV sequence in the context of RhPV genome, are manipulated to be separated from other portions of the RhPV genome, or to be recombined with other sequences that may or may not be originated from the same source.

The practices of this invention are hereinafter described in detail with respect to a nucleic acid sequence isolated from 5′-UTR of Rhopalosiphum padi virus having a promotor activity and a polynucleotide sequence set forth in SEQ ID NO: 1 of the sequence listing, or a fragment thereof; and to the use of the isolated RhPV nucleic acid sequence for constructing a viral vector for gene delivery in an insect expression system.

The inventors of the present invention unexpectedly identify that the internal ribosomal entry site (IRES) element in the long, highly structured 5′-untranslated region (5′-UTR) of Rhopalosiphum padi virus may act, not only as an IRES element for initiating cap-independent translation, but also as a promotor for initiating transcription of a nucleic acid operably linked thereto. The promotor activity of the IRES element of RhPV is shown to be active in an insect system, whereas the IRES activity is active in mammalian cells. In one example, the IRES RhPV is identified to contain at least six TAAG motifs within its structure. The TAAG motifs within a gene have been demonstrated to be essential for transcriptional initiation of that gene. Thus, it is believed that the 6 TAAG motifs in the isolated nucleic acid of RhPV or IRES RhPV are responsible for its promotor activity.

The identified promotor comprises a polynucleitide sequence set forth in SEQ ID NO: 1 of the sequence listing, fragments thereof, and derivatives thereof, such as deletion constructs. The present invention therefore encompasses a polynucleotide sequence, which have one or more nucleotide deleted, inserted or substituted, provided that the polynucleotide sequence has sufficient activity in initiating translation. The present invention therefore extends to polynucleotide sequences capable of hybridizing to the polynucleotide sequence set forth in of SEQ ID NO:1, and which retain appropriate functional activity. Hybridization may be assessed using standard techniques as described in a basic textbook (Sambrook et al., Molecular Cloning 3rd Ed., Cold Spring Harbor Laboratory Press (2001)). A hybridising polynucleotide preferably hybridizes under stringent conditions to the target polynucleotide, the stringency conditions being selected to reflect a degree of substantial identity as discussed herein. For example, the conditions preferably give hybridization when there is about 80% identity or more, such as from 85% identity, from 90% identity or from 95% identity or more. Stringency, as it is commonly used in the art, refers to salt concentration ordinally be less than about 750 mM NaCl and 75 mM trisodium citrate, preferably less than about 500 mM NaCl and 50 mM trisodium citrate, and most preferably less than about 250 mM NaCl and 25 mM trisodium citrate. Stringent temperature conditions will ordinarily include temperature of at least about 30° C., more preferably of at least about 37° C., and most preferably of at least about 42° C. Varing additional parameters such as hybridization time, concentration of detergent (such as sodium dodecyl sulphate (SDS) and others are well known to those skilled in the art. A preferred RhPV promotor sequence is a nucleotide sequence that is at least 80% identical to the polynucleitide sequence set forth in SEQ ID NO: 1, and more preferably is at least 81%, 82%, 83%, 84%, 85%, 86%, 87%, 88%, 89%, 90%, 91%, 92%, 93%, 94%, 95%, 96%, 97%, 98%, 99% or 100% identical to the polynucleitide sequence set forth in SEQ ID NO: 1. Percentage of identity is a measure of the number of identical nucleotides in an uninterrupted linear sequence of nucleotides when compared to a target nucleotide sequence of specified jength. As used herein, “identity” of a nucleotide sequence means that the compared nucleotide residues in two separate sequences are identical. Thus, 100% identity means, for example, that upon comparing 50 sequential nucleotides in two different molecules, both residues in all 50 pairs of compared nucleotides are identical.

There are various methods known to those of skill in the art, which may be used to prepare or isolate the RhPV nucleic acid having a promotor activity. For example, the IRES RhPV nucleic acid can be isolated from 5′-UTR of Rhopalosiphum padi virus. See Sambrook et al (supra) for a description of techniques for the isolation of DNAs related to DNA molecules of known sequence. Alternatively, the IRES RhPV nucleic acid can also be synthesized by a known method that utilizes a polymerase chain reaction (PCR) method based on the sequence information set forth in SEQ ID NO: 1 of the sequence listing. Such a method can be used to amplify nucleic acid sequences from mRNA, from cDNA, and from genomic libraries or cDNA libraries. These methods can be easily carried out by a skilled person in the art according to a basic textbook (Sambrook et al., supra) and the like.

The isolated or synthesized nucleic acid of RhPV or IRES RhPV can be used as a promotor for constructing a vector for initiating transcription of a nucleic acid operably linked thereto. This invention thus provides a vector comprising a promotor sequence isolated from RhPV, the promotor sequence is capable of initiating transcription of an operably linked nucleic acid in insect cells, and has a polunecleotide sequence at least 80%, preferably 81%, 82%, 83%, 84%, 85%, 86%, 87%, 88%, 89%, 90%, 91%, 92%, 93%, 94%, 95%, 96%, 97%, 98%, 99% or 100% identical to the sequence set forth in SEQ ID NO: 1 of the sequence listing. In one preferred example, a promotorless bicistronic vector, particularly, a baculoviral vector, was constructed and transfected into an insect cell expression system such as S. frugiperda IPBL-Sf21 insect cells; and the promotor activity of the IRES RhPV sequence is confirmed by the green fluorescence emited from the expressed enhanced green fluorescent protein (EGFP), whose gene is operably linked to and driven by the isolated or synthesized IRES RhPV sequence. Construction of vectors comprising the isolated or synthesized nucleic acid of RhPV or IRES RhPV can be easily carried out by a skilled person in the art according to a basic textbook such as the above Molecular Cloning. The vector comprising the IRES RhPV having a promoter activity may therefore be used as a tool for delivering desired genes or initiating desired gene expression in an insect system.

To provide those skilled in the art the tools to use the present invention, the isolated or synthesized RhPV nucleic acids, vectors and insect cells of the invention are assembled into kits. The components included in the kits are the isolated or synthesized RhPV nucleic acids, viral vectors, enzymatic agents for making the recombinant viral constructs, cells for amplification of the viruses, and reagents for transfection and transduction into the target cells, as well as description in a form of pamphlet, tape, CD, VCD or DVD on how to use the kits.

The following examples are provided to illustrate the present invention without, however, limiting the same thereto.

### EXAMPLES

### Example 1

### Building Plasmid Constructs and Recombinant Vectors

1.1 pD-E

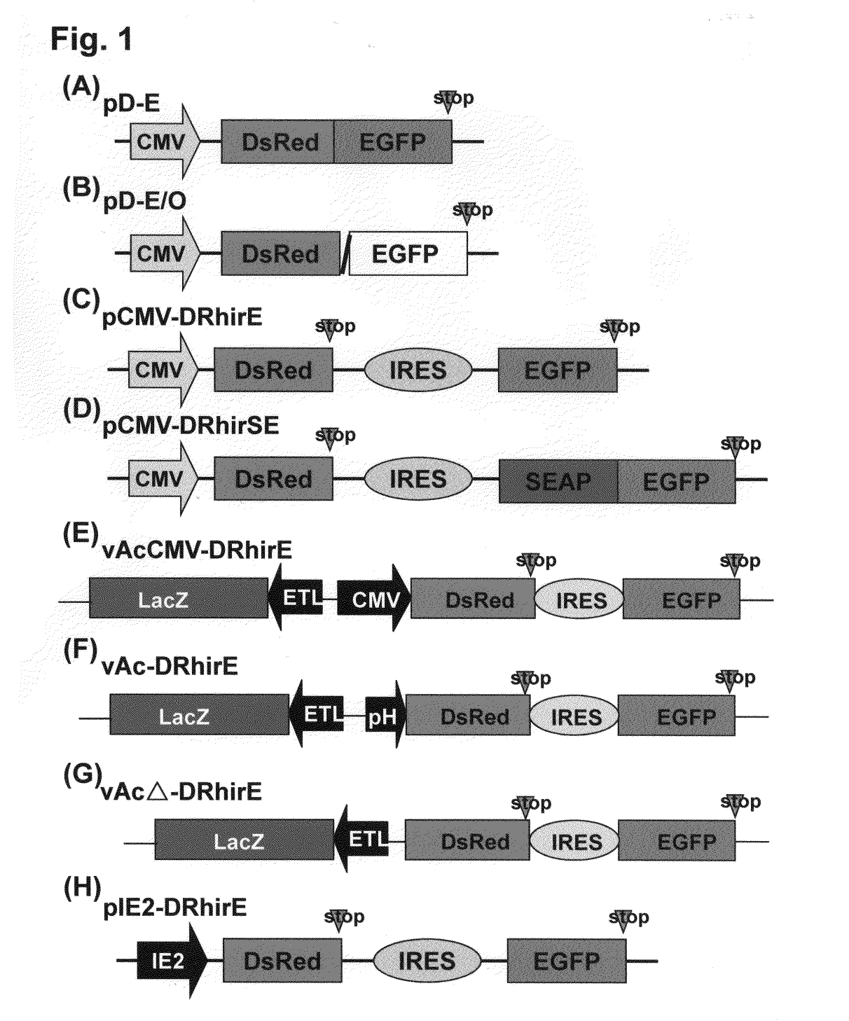

pD-E was generated by first digesting the plasmid pDsRed1-N1 (ClonTech) with NheI and EcoRI to release the DsRed gene fragment, and followed by cloning the released DsRed gene fragment into the NheI and EcoRI sites of the pEGFP-C1 plasmid (ClonTech). The resulting plasmid was used as a positive control, with the DsRed and EGFP fused in frame (FIG. 1A).

1.2 pD-E/O

pD-E/O was prepared by first digesting the plasmid pDsRed1-N1 (ClonTech) with EcoRI, and then followed by treatment with a mung bean nuclease (New England Biolab, NEB) before the ligation reaction took place. Therefore, in pD-E/O (FIG. 1B), the DsRed gene was not fused in-frame with the EGFP gene.

1.3 pCMV-DRhirE

pCMV-DRhirE was constructed by digesting pBacDRhirE plasmid (Chen et al., Biochem and Biophys Res Commun 335 (2005) 616-623) with NheI and is SalI and subcloned the 2.6-kb DsRed-IRES-EGFP DNA fragment into an NheI- and SalI-digested pEGFP-C1 plasmid (ClonTech, Mountain View, Calif. USA). The resulting plasmid was named pCMV-DRhirE (FIG. 1C). Briefly, to construct pCMV-DRhirE, the CMV promoter was first amplified from the pEGFP-C1 plasmid by PCR with the forward primer, 5′-GCCGATATCTAGTTAATTAATAGTAATC-3′ (SEQ ID NO: 2, the EcoRV site is underlined), and the reverse primer, 5′-AGCGCTAGCGGATCTGACGGTTCACTAAA-3′ (SEQ ID NO: 3, the NheI site is underlined). Then, the PCR product, the CMV promoter fragment, was cloned into the EcoRV- and NheI-digested pBacDRhirE plasmid, replacing the polyhedrin promoter. Hence, pCMV-DrhirE contains in sequence the DsRed reporter gene, the IRES sequence of Rhopalosiphum padi virus (RhPV) and EGFP reporter gene.

1.4 pBacADRhirE

pBacΔDRhirE, a promoterless vector, was generated from pCMV-DrhirE of example 1.3 by first digesting with EcoRV and NheI then treated with mung bean nuclease before the blunt ligation reaction.

1.5 pIE2-DRhirE

pIE2-DrhirE was constructed by subcloned the 2.6-kb DsRed-IRES-EGFP DNA fragment of example 1.3 into an SpeI- and XhoI-digested pIB vector (Invitrogen, Carlsbad, Calif., USA), and a baculovirus-independent, insect cell transient expression vector, pIE2-DRhirE (FIG. 1H) was obtained.

1.6 pCMV-DRhirSE

pCMV-DrhirSE was constructed by fusing the secretory alkaline phosphatase (SEAP) with the EGFP gene in the pCMV-DRhirE of example 1.5. Briefly, the SEAP gene fragment was first amplified from the pGS-HCV plasmid by PCR with the forward primer, 5′-ATATAAGATCTCCACCATGCTGCTGCTGCTGCTGCTGCTGGG-3′ (SEQ ID NO: 4, the BglII site is underlined), and the reverse primer, 5′-AATTCAGATCTGGTGTCTGCTCGAAGCGGCCGGC-3′ (SEQ ID NO: 5, the BglII site is underlined). Then, the 1.6-kb, BglII-digested SEAP gene fragment was subcloned into the Barn HI-digested pBacDRhirE plasmid and fused in-frame with the N-terminal of the EGFP gene.

### 1.7 Production of Recombinant Vectors

Using cell fectin (1 μl), Sf21cells (2×10⁵ cells per well in a 24-well plate) were cotransfected with the linearized viral DNA Bac-N-Blue (0.25 μg, Invitrogen) and 0.8 μg of one of the transfer vectors, either pCMV-DrhirE of example 1.5 or pBacΔ-DrhirE of example 1.4. The resulting viruses were respectively named vAcCMV-DRhirE (FIG. 1E) and vAcΔ-DRhirE (FIG. 1G). For the Bac-N-Blue viral DNA containing the LacZ gene controlled by the ETL promoter, the recombinant virus was identified by X-gal staining in accordance with the manufacturer's protocol. The recombinant viruses were selected and purified by a series of three end-point dilutions. Sf21 monolayers were used for virus propagation, and all viral stocks were prepared and titers determined according to the end point dilution^{]}. And the recombinant viruses, vAc-DCrirE and vAc-DRhirE (FIG. 1F) was used in the Northern blot analysis. vAc-DCrirE and vAc-DRhirE containing the DsRed and EGFP genes flanking the IRES derived from the IGR IRES sequence of the Cricket paralysis virus (CrPV) and RhPV IRES, respectively. These two recombinant baculoviruses were prepared and processed as described before (Wu T Y et al., FEBS Letters 581 (2007) 3120-3126).

### Example 2

### Confirmation of RhPV IRES Activity in Mammalian Cells

The IRES activity of the isolated RhPV sequence (i.e., sequence set forth in SEQ ID NO: 1) was confirmed by observing the reporter genes under a fluorescence microscope and by protein activity measurement.

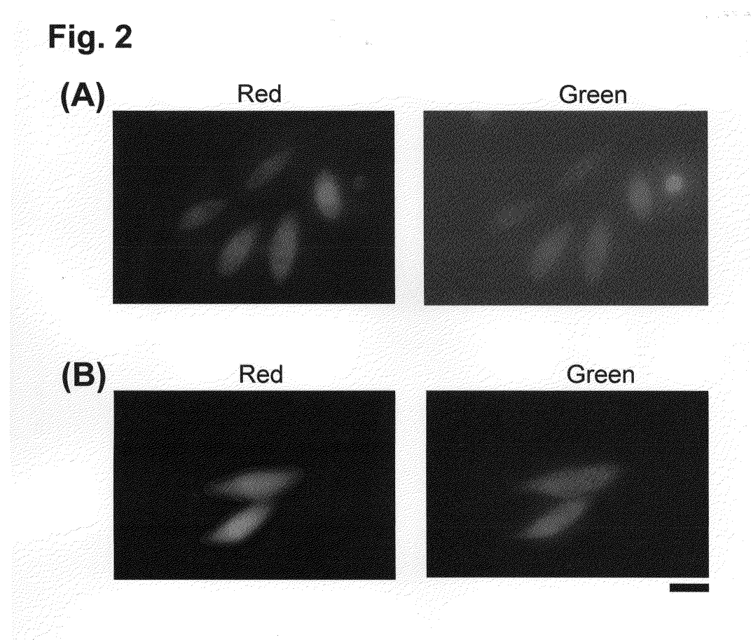

Fluorescence observation In this study, mammalian CHO cells were transiently transfected with either pD-E of example 1.1 or pCMV-DhirE of example 1.3. In pD-E transfected cells, since the two reporter genes, DsRed and EGFP were fused in frame, hence both the red and green fluorescence were revealed in the same cells (FIG. 2A). Similarly, pCMV-DrhirE transfected CHO cells also revealed both red and green fluorescences (FIG. 2B). However, CHO cells transfected with the plasmid pD-E/O (FIG. 1B) in which the DsRed gene was not in-frame-fused with the EGFP gene only revealed the red fluorescence but without green fluorescence (data not shown). This result confirmed that the RhPV IRES is capable of mediate cap-independent translation in CHO cells.

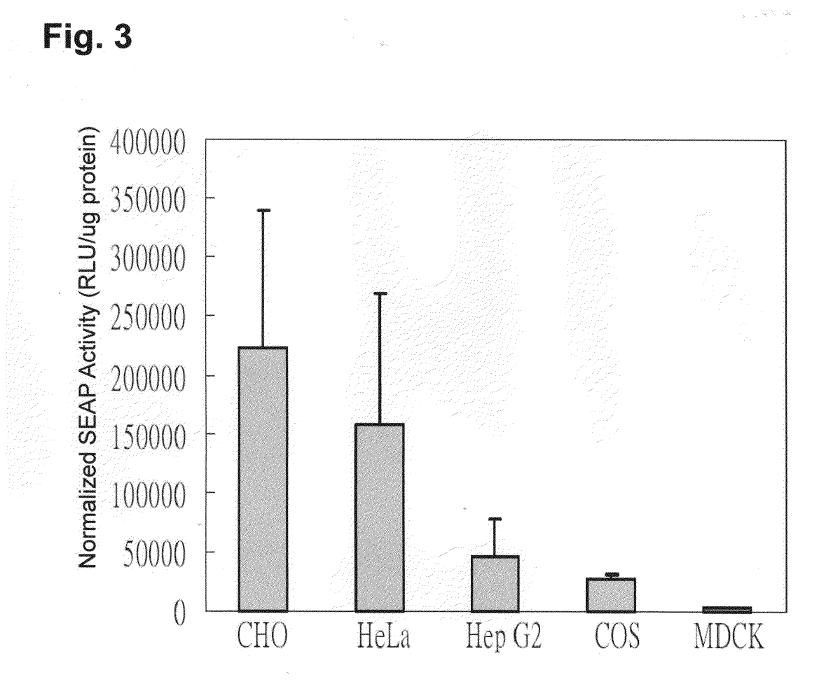

Measurement of SEAP activity Since the pCMV-DRhirSE plasmid of example 1.6 contains a SEAP reporter gene fused in-frame with an EGFP fluorescent gene; therefore, the RhPV IRES activity can be easily and sensitively monitored by measuring the SEAP from the medium. Briefly, mammalian cells, including CHO-K1; COS1; HeLa; HepG2; MDCK and U2OS cells, were transfected with plasmids of example 1.6 by use of the Lipofectin reagent (Invitrogen). Cells (at 9×10⁴˜10⁵ per well) were plated onto 24-well plates. Before transfection, cells were repeatedly washed with serum-free media to remove any traces of sera. One microgram of pCMV-DRhirSE plasmid of example 1.6 (FIG. 1D) was diluted in 200 μl of serum-free DMEM or MEM medium, and 1 μl of the Lipofectin reagent was added. The DNA-Lipofectin mix was incubated for 15 min for DNA-Lipofectin complex formation. Then, the DNA-Lipofectin complex solution was transferred to the cells, at a total volume of 0.5 ml by adding serum-free medium. After 12 h, the medium was removed, and 1 ml of fresh medium with 10% fetal bovine serum was added. Two days post-transfection, supernatants were harvested and analyzed for SEAP activity. The SEAP activity in the culture media was measured using BD Great EscApe™ SEAP detection kits (ClonTech). The chemiluminescent intensities reflecting relative SEAP activities were detected with a chemical luminescence counter (Mithras LB 940, Berthold Technolies). Results were illustrated in FIG. 3.

For all six cell lines transiently transfected with the pCMV-DRhirSE plasmid of example 1.6, all transfected cells expressed cap-dependent translation of the DsRed gene under a fluorescence microscope (data not shown). However, after normalized with DsRed fluorescence, the SEAP-EGFP activities in the culture medium differed among these cell lines (FIG. 3). The RhPV IRES exhibited significantly higher translation efficiency in CHO and HeLa lines than that in HepG2, COS-1, and MDCK cell lines. This result suggests the RhPV IRES may not function equally in these tested mammalian cells, although it has been proven to be a cross-kingdom IRES. However, it does not exclude to the possibility that the CMV promoter activity may differ in these tested cell lines.

### Example 3

### RhPV IRES Has a Cryptic Promoter Activity in Baculovirus-Infected Sf21 Cells

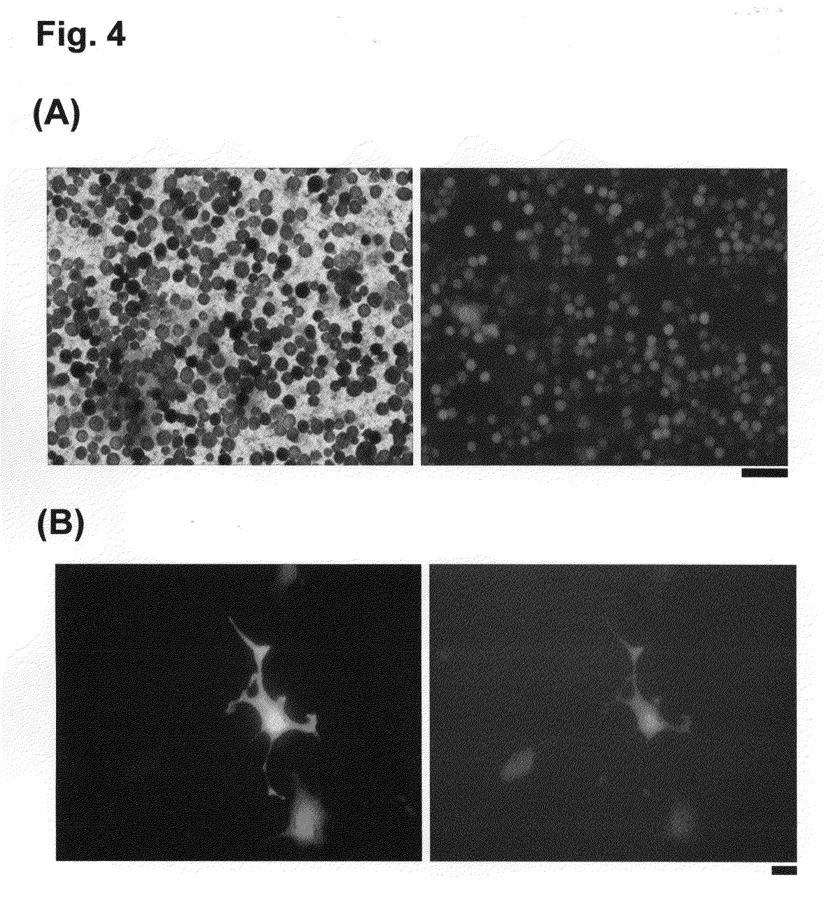

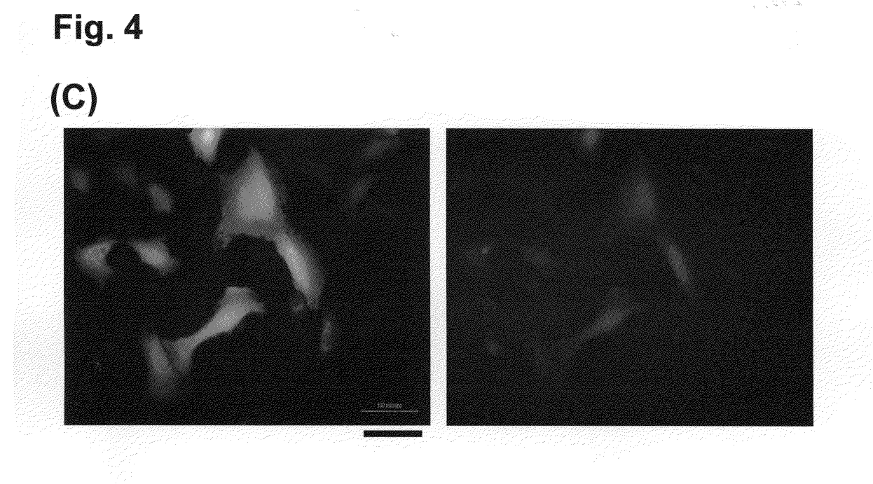

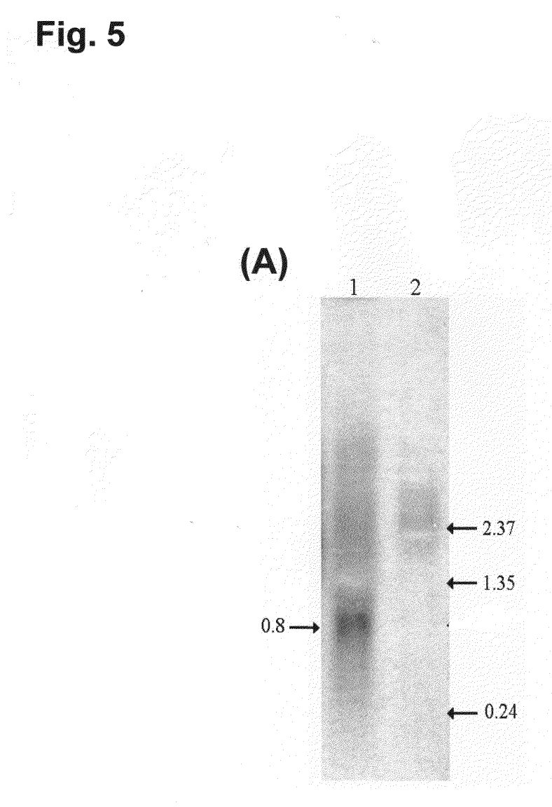

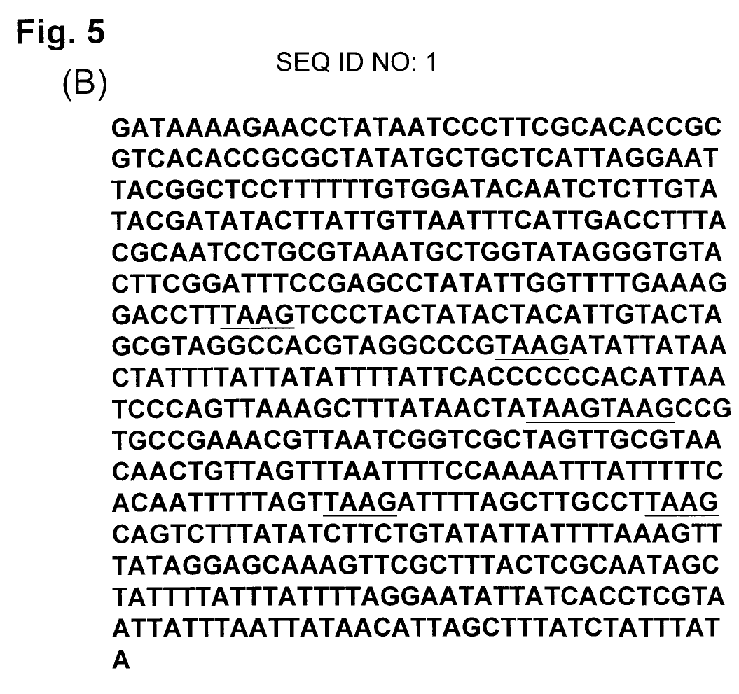

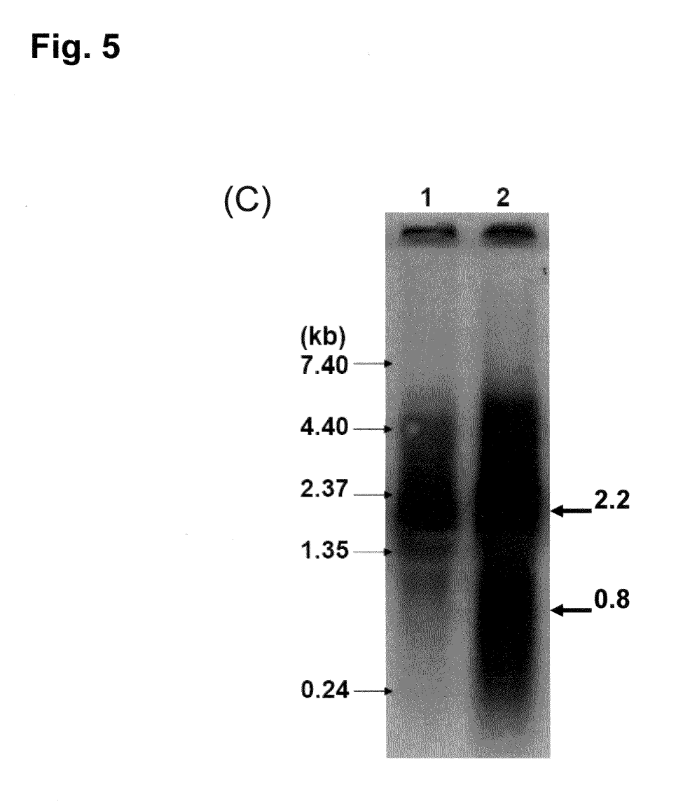

Since it is reported that CMV promoter does not function in insect cells (Wu T Y et al., J. Biotechnol. 2000 80: 75-83), hence the vaculovirus vector (i.e., vAcCMV-DrhirE (FIG. 1E)) infected recombinant virus prepared in accordance with procedures described in example 1.7 was isolated by X-gal staining for the vAcCMV-DRhirE genome containing the LacZ gene driven by the ETL promoter (FIG. 4A, left). As expected, the vAcCMV-DRhirE-transduced COS-1 cells revealed both red and green fluorescence under a fluorescence microscope (FIG. 4B). The inventors previous studies also showed that the bone derived cell lines are more accessible to baculovirus than COS-1 cells (Liu Y K et al., Acta Pharmacol Sin 27 (2006) 321-327), therefore, the bone derived cell line, U2OS, was transduced with vAcCMV-DRhirE. FIG. 4C shows that both red and green fluorescence were expressed in the transduced cells. These results indicate that the RhPV IRES is functional in mammalian cells either by a plasmid vector or by a recombinant viral genome. Interestingly, vAcCMV-DRhirE-infected Sf21 cells revealed green fluorescence (FIG. 4A, right), in the absence of red fluorescence (data not shown). These unexpected results imply that the RhPV IRES may possess a cryptic promoter activity, or that DsRed-RhPV IRES-EGFP bicistronic mRNA undergoes RNA cleavage in vAcCMV-DRhirE-infected Sf21 cells. To clarify these questions, a Northern blot analysis and a promoterless assay were performed. Results were illustrated in FIG. 5.

Northern Blot Analysis Briefly, an EGFP gene fragment (366 bp) was amplified by PCR from the pBac-DRhirE plasmid using the primer set, EGFP-F (5′-ACGACTTCTTCAAGTCCGCC-3′, SEQ ID NO: 6) and EGFP-R (5′-TGCTCAGGTAGTGGTTGTCG-3′, SEQ ID NO: 7). The fragment was then cloned into a pGEM-T Easy Vector (Promega), which contains T7/SP6-opposed promoters. DIG-RNA probes were prepared by in vitro transcription with a commercial kit (DIG-RNA labeling kit, Roche) according to instructions provided by the manufacturer. Total RNA transcripts were extracted from vAcCMV-DRhirE-, vAc-DCrirE- and vAc-DRhirE-infected Sf21 cells at 4 days post-infection (dpi) and also from uninfected Sf21 cells. Extracts were electrophoresed in a 1% agarose gel containing formaldehyde, blotted onto a nylon membrane (Hybond-N, Amersham), and probed with the EGFP probe according to the standard procedure of Sambrook et al. (Molecular cloning: a laboratory manual (2001), 3rd ed. Cold Spring Harbor Laboratory Press, Cold Spring Harbor, N.Y.) Standard chemiluminescent detection was performed according to the manufacturer's instructions (Roche), and the blot was exposed to x-ray film (Kodak XAR-5).

FIG. 5A indicates that a transcript of around 0.8 kb, corresponding to the EGFP gene transcript, was detected in vAcCMV-DRhirE-infected Sf21 cells. However, the predicted size of the bicistronic RNA transcript (about 2 kb) containing the DsRed gene (680 bp), the RhPV IRES (579 bp), and the EGFP gene (798 bp) in vAcCMV-DRhirE-infected Sf21 cells was not found. This result implied that the CMV promoter did not mediate the transcription of the bi-cistronic transcript in the baculovirus infected Sf21 cells and the expression of green fluorescence proteins may be driven by a promoter within the RhPV IRES. Six TAAG motifs are identified in the DNA sequence that corresponds to the RhPV IRES (FIG. 5B). TAAG sequences are relatively rare in the AcMNPV genome and are found primarily in late or very late promoter regions. Thus, the six TAAG motifs in the DNA sequence of RhPV 5′ IRES may be responsible for the promoter activity in baculovirus-infected Sf21 cells. These results suggest that vAc-DRhirE (see FIG. 1F), the bicistronic baculovirus vector that has a DsRed and an EGFP gene flanking the RhPV IRES controlled under the polyhedron promoter, infected insect cells would generate two transcripts: one containing the bicistronic transcript and the other containing only the EGFP transcript. To test this hypothesis, another Northern blot analysis was performed with a DIG-labeled GFP-specific probe and both a 2 kb and 0.8 kb transcripts were detected in the vAc-DRhirE infected Sf21 cells (FIG. 5C, lane 2). In contrast, in the cell lysates of the vAc-DCrirE-infected Sf21 cells, a species of RNA with a size of about 2 kb was detected (FIG. 5C, lane 1). These Northern blots analysis suggested that a cryptic promoter may occur in the RhPV IRES and mediates the EGFP gene transcription in the baculovirus infected Sf21 cells.

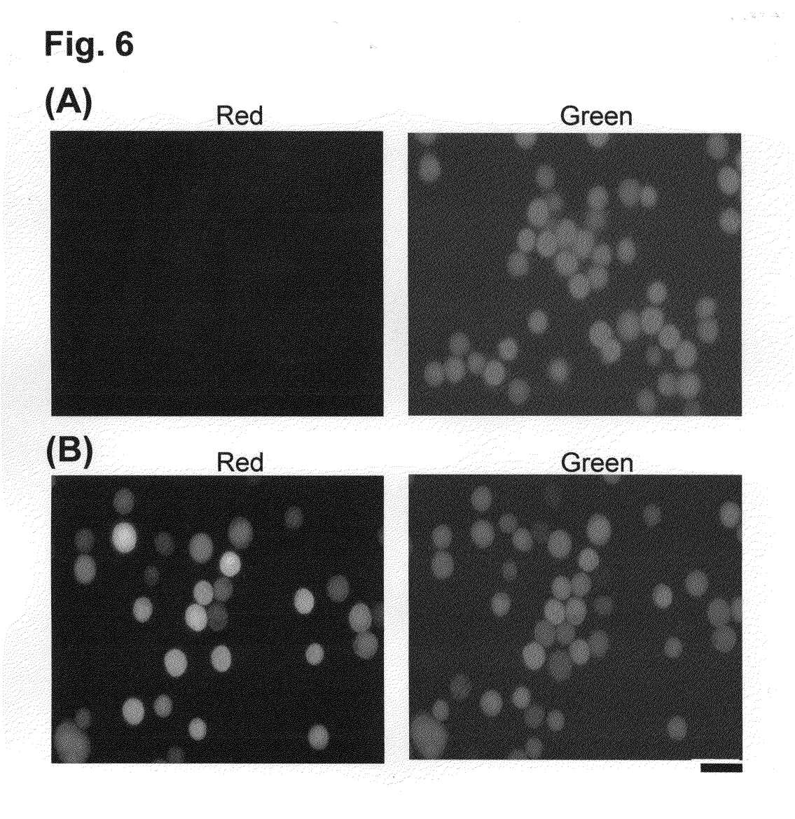

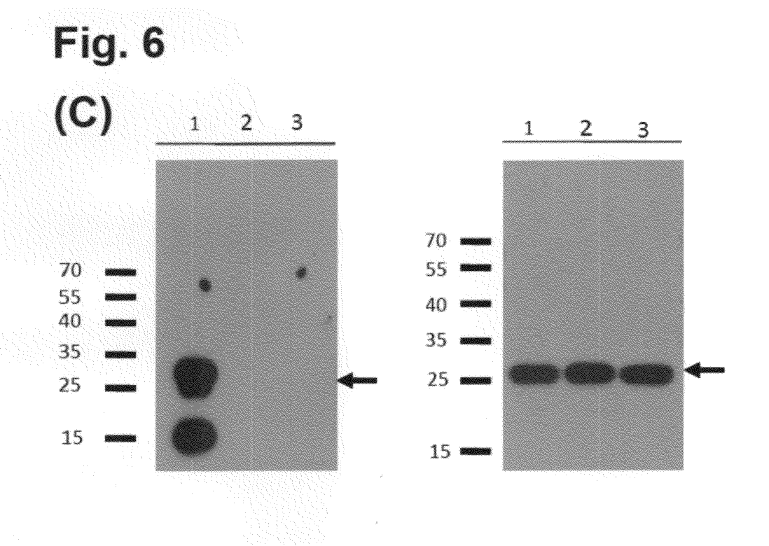

Promoterless Assay Briefly, a promoterless bicistronic vector, pBacΔ-DrhirE, formed by simply removing the CMV promoter from pCMV-DRhirE, was constructed in accordance with the procedures described in example 1.4, and the resulting recombinant baculovirus was named vAcΔ-DRhirE (FIG. 1G). This promoterless recombinant virus should not generate bicistronic mRNAs after infection of Sf21 cells, due to the lack of a CMV or a polyhedrin promoter. Thus, this promoterless recombinant baculovirus should not possess the RhPV IRES activity, and any expression of the second cistron EGFP gene from vAcΔ-DRhirE-infected Sf21 cells should be due to the existence of a promoter activity in the RhPV IRES sequence. FIG. 6A illustrates that vAcΔ-DRhirE-, like vAcCMV-DRhirE-, the infected Sf21 cells revealed only green fluorescence and no red fluorescence. In contrast, vAc-DRhirE-infected Sf21 cells in which the bi-cistronic construct was made from polyhedrin promoter expressed both green and red fluorescence (FIG. 6B). Western blot analysis also confirmed the expression of fluorescent proteins in these baculovirus infected Sf21 cells. Results were illustrated in FIG. 6C.

Western Blot Analysis Proteins were separated by SDS-PAGE on a mini Protein III system (Bio-Rad, Hercules, Calif., USA). After SDS-PAGE fractionation, proteins were electrotransferred onto a PVDF membrane (polyvinylidene difluoride, Millipore, Bedford, Mass., USA). The resulting membrane was blocked with Tris-buffered saline [TTBS; 100 mM Tris (pH 7.4), 100 mM NaCl, and 0.1% Tween 20] containing 5% (v/v) non-fat dry milk at room temperature for 1 h with gentle shaking. Subsequently, the membrane was incubated with a 1:2000-diluted anti-EGFP or anti-DsRed antibodies (ClonTech) in TBS with 0.5% (v/v) non-fat dry milk at 4° C. overnight. Unbound antibodies were removed by three washes each of 5min in TTBS buffer at room temperature with shaking. Then the membrane was incubated with 1:2500-diluted horseradish peroxidase (HRP) conjugated secondary antibodies (Chemicon) for 1 h at room temperature. The HRP on the membrane was detected by an enhanced chemiluminescence kit (Pierce, Rockfold, USA) following the protocol provided by the manufacturer. As shown in FIG. 6C, the DsRed protein only expressed in the vAc-DRhirE-infected Sf21 cells but the EGFP protein can be detected in vAc-DRhirE-, vAcCMV-DRhirE- and vAcΔ-DRhirE-infected Sf21 cells.

The combined results of the Northern and the Western blot analysis demonstrated that the RhPV IRES is capable of mediating gene expression in baculovirus infected Sf21 cells through a cryptic promoter. However, neither green nor red fluorescence appeared in CHO and U2OS cells transiently transfected with the promoterless pBacΔ-DRhirE bicistronic vector or transduced with vAc-DRhirE (data not shown). These results indicated that this cryptic promoter, like the TAAG-containing baculovirus late promoter, depends on baculovirus early gene expression. Thus, when Sf21 cells were transfected with pCMV-DRhirE (FIG. 1C), neither the red fluorescence nor the green fluorescence were observed (data not show).

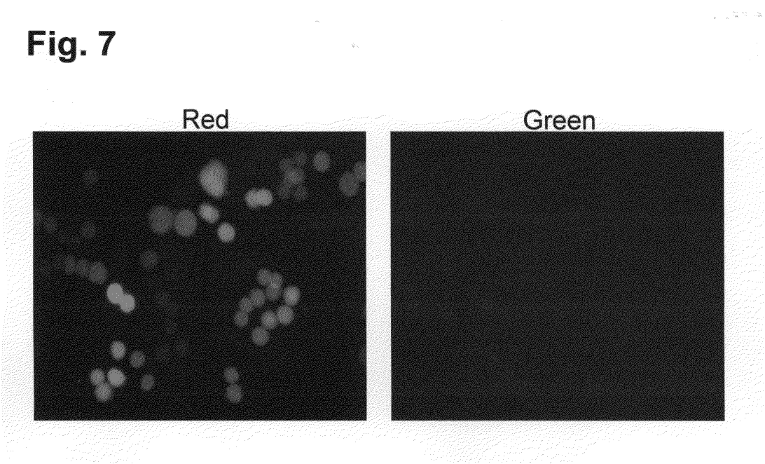

To further confirm this observation and rule out any experimental bias (e.g., failure plasmids transfection can also get this result), another construct, pIE2-DRhirE (FIG. 1H) is generated, to analyze RhPV IRES activity in insect cells without baculovirus infection. In the pIE2-DRhirE transfected Sf21 cells, the transcription of the bi-cistronic transcripts, containing the DsRed and EGFP open reading frame sequence flanking the RhPV IRES, is mediated by the baculovirus independent immediately early promoter, ie2 promoter. It is found that only the cap-dependent translation of DsRed was observed, and the green fluorescence was barely detected under fluorescent microscopy (FIG. 7). Thus, the expression of the green fluorescence proteins EGFP from the vAc-DRhirE infected Sf21 cells (FIGS. 6B and 6C) is mostly mediated by the cryptic promoter activity of RhPV IRES rather than the cap-independent, IRES mediated translation activity. These results were also consistent with the observation of to the Northern blot presented in the FIG. 5C. Taken together, these results confirm that the DNA sequence of RhPV IRES exhibits a cryptic promoter in baculovirus-infected Sf21 cells but not in mammalian cells.

While the foregoing is directed to embodiments of this invention, other and further embodiments of the invention may be devised without departing from the basic scope thereof, and the scope thereof is determined by the claims that follow.

1-6. (canceled)

7. A vector comprising an isolated or synthesized promotor sequence that is capable of initiating transcription of an operably linked nucleic acid sequence in insect cells, wherein the promotor sequence is at least 90% identical to the isolated or synthesized Rhopalosiphum padi virus (RhPV) nucleic acid of SEQ ID NO: 1.

8. The vector of claim 7, wherein the vector is a baculovirus.

9. The vector of claim 7, wherein the insect cells are S. frugiperda IPBL-Sf21 insect cells.

10. The vector of claim 7, wherein the promotor sequence comprises 6 TAAG motifs.

11. The vector of claim 7, wherein the promotor sequence has an IRES activity in insect cells, plant cells or mammalian cells.
